(19) United States
(12) Patent Application Publication
(10) Pub. No.: US 2007/0264332 A1
(43) Pub. Date: Nov. 15, 2007

(54) Sustained Release Pharmaceutical Formulation

(76) Inventors: Jose Gutierrez-Rocca, Miami, FL (US); Saul Rios, Miramar, FL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/782,886

(22) Filed: Jul. 25, 2007

Related U.S. Application Data
(63) Continuation of application No. 10/086,059, filed on Feb. 27, 2002.
(60) Provisional application No. 60/271,828, filed on Feb. 27, 2001.

(51) Int. Cl. A61K 9/22; A61K 31/135; A61K 31/166; A61K 31/40; A61K 31/4164; A61K 31/437; A61K 31/455; A61K 31/495; A61K 31/496; A61K 31/522; A61K 31/554

(52) U.S. Cl. 424/468; 514/211.05; 514/254.05; 514/255.04; 514/263.34; 514/300; 514/356; 514/398; 514/423; 514/619; 514/620; 514/646; 514/657

(57) ABSTRACT

A sustained release pharmaceutical formulation is disclosed. The formulation comprises a water soluble medicament and a polymer mixture comprising a first component of about 80 weight percent polyvinylacetate combined with about 20 weight percent polyvinyl pyrrolidone; of the total weight of the first component, combined with a second component of a cellulose ether polymer.

This application claims benefit of provisional application Ser. No. 60/271,828 which was filed on Feb. 27, 2001 and which is incorporated by reference hereinto in its entirety.

### BACKGROUND OF THE INVENTION

1. Field of the Invention

This invention relates to a controlled release pharmaceutical formulation, and, more particularly, to a formulation comprising (a) a water soluble medicament and (b) a mixture of polymers comprising a first mixture of polyvinyl acetate and polyvinyl pyrrolidone combined with a cellulose ether.

2. Description of the Related Art

Many water soluble medicaments are poorly absorbed or transported in the body of a patient being treated, i.e. a human being or another animal, possibly due to a combination of several factors including large molecular size, ionization, high surface charge, enzymatic and chemical instability and low permeability of absorption barriers in the patient's body. In numerous therapies, drug dosimetry is increased by orders of magnitude to achieve minimum systemic concentrations required for efficiency.

The clinical and pharmaceutical chemistry sciences, in an attempt to accomplish the highest level of therapeutic benefit for those drugs or compounds, have resorted to chemical modifications as a principal mode for improving biological activity of these drugs in the body of the patient. The mode of drug administration to the body has also gradually expanded from oral and parenteral to transdermal, rectal and the pulmonary routes of administration, i.e., nose and lung. Success and achievement with these drug delivery approaches are mixed largely due to lack of acceptance of the newer, complex molecules that must be used for treating difficult diseases of the body, e.g., infections, malignancies, cardiovascular, endocrine, neurologic diseases, and a variety of immunologically compromised diseases, like AIDS.

Accordingly, what is desired and needed is a formulation system comprising an active pharmaceutical ingredient (“API”) that is stable and protected by a rate-limiting carrier, easily manufactured and therapeutically effective when administered to a patient, i.e., a human being or another animal.

### SUMMARY OF THE INVENTION

This invention relates to a modulated release or sustained release pharmaceutical formulation, and, more particularly, to such a formulation comprising a water soluble medicament combined with a carrier comprising a mixture of polymers.

### DETAILED DESCRIPTION OF THE INVENTION

The present invention relates to a sustained, modulate or controlled pharmaceutical formulation comprising (1) a selected water soluble medicament or drug, (2) a suitable construct with which the drug is associated, i.e. is embedded, therewith or being part of the construct. By embedded is meant that the drug is homogeneously distributed throughout the construct. The construct provides a modulated release of the associated, e.g. embedded, drug to the body of a patient, e.g. a human being or another animal, when the construct is administered, e.g. orally, to the patient.

The formulation is ideally intended to be administered to the patient in an oral dosage form, typically comprising but not limited to, a tablet, a capsule or caplet.

Suitable therapeutic medicament categories of drugs or medicaments are those which are water soluble or water soluble to some degree and which can be administered to a patient orally, and include cardiovascular drugs, antiallergics, analgesics, bronchodialators, antihistamines, antitussives, antifungals, antivirals, antibiotics, antidepressants and other pain medicaments, antiinflamatories, analgesics etc. Particularly suitable medicaments include a pharmaceutically acceptable acid addition salt of hydroxyzine; a pharmaceutically acceptable acid addition salt of metoprolol e.g. tartrate; succinate, fumarate; niacin; caffeine; theophylline; a pharmaceutically acceptable acid addition salt of diltiazem; a pharmaceutically acceptable acid addition salt of albuterol; a pharmaceutically acceptable acid addition salt of metformin; a pharmaceutically acceptable acid addition salt of metronidazole; a pharmaceutically acceptable acid addition salt of metoclopramide; a pharmaceutically acceptable acid addition salt of ranitidine; a pharmaceutically acceptable acid addition salt of captopril; pharmaceutically acceptable acid addition salt of nefazodone; pharmaceutically acceptable acid addition salt of zolpidem; pharmaceutically acceptable acid addition salt of sertraline; pharmaceutically acceptable acid addition salt of labetalol; pharmaceutically acceptable acid addition salt of atenolol.

For purposes of the formulations of this invention, which are illustratively, typically intended for tabletting into a tablet unit dosage form, the medicament, e.g. water soluble biotherapeutic medicament or drug, is associated with the construct carrier with which it is destined to be combined. By “associate” or “associated” is meant that the medicament is embedded within a matrix or a part of the matrix along with the other component making up the construct or is homogeneously distributed within the carrier matrix, or is on the surface of the carrier matrix.

A suitable construct is selected. Such a construct is one which will incorporate or embed the selected medicament and provide a controlled or modulated release of the medicament therefrom into the gastrointestinal tract to be carried to the sites of action or application in the body.

A suitable carrier construct comprises a mixture of polymers. The polymer mixture comprises a mixture having a first component combined with a second component. The first component of the polymer mixture comprising about 80 weight percent polyvinyl acetate combined with about 20 weight percent of polyvinyl pyrrolidone. The second component of the polymer mixture comprising a cellulose ether polymer. The first component of the polymer mixture further comprising an amount from about 20 weight percent to about 90 weight percent of the total weight of the formulation or construct and the second component further comprising an amount from about 2 weight percent to about 60 weight percent of the total weight of the formulation or construct, with the remainder comprising the water soluble medicament or mixture of medicaments, alone or with suitable excipients, as discussed hereafter.

A suitable cellulose ether polymer is one having a structure 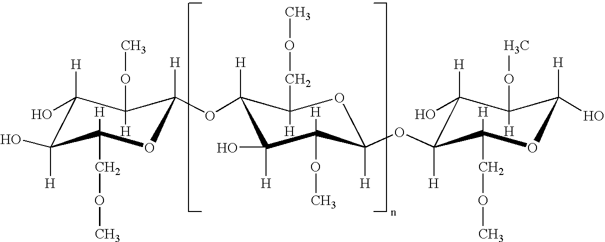
These polymers are commercially available from the Dow Chemical Company, Midland, Mich., under the Tradename “METHOCEL”, e.g. METHOCEL A SERIES and from ShienEtsu Chemical Co. Ltd., Niigata, Japan. Another suitable cellulosic polymer is a hydroxypropoxyl methyl cellulose polymer having a structure, 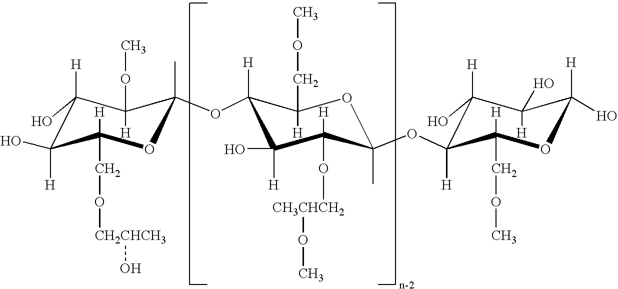
These polymers are commercially available from the Dow Chemical Company, Midland, Mich., under the tradename “METHOCEL”, e.g. METHOCEL E, F, J, K SERIES and from ShienEtsu Chemical Co. Ltd., Niigata, Japan, e.g. Metolose and LH series. Preferably the formulation comprises a mixture of at least two of the foregoing components.

The dosage form, e.g. a tablet, utilizes the formulation, i.e. the construct or the matrix having the medicament associated therewith.

The water soluble medicaments, of the formulation and the sustained/prolonged release dosage form of the present invention, comprise a group of pharmaceutically active drugs having a solubility greater than 1 gram (g) of drug in about 1000 milliliters (ml) of water (from very soluble to slightly soluble)- to 1 gram(g) compounds that will require less than 1000 ml to dissolve the drug.

Some drugs having such a solubility in water include, but are not limited to, metoprolol, nefazodone, zolpidem, sertraline, labetatol, atenolol, metformin, niacin, caffeine and theophylline. Drugs not having a solubility in water greater than about 1 g/1000 ml of water may be solubilized by the addition of a solubilizing agent in the matrix e.g. surfactants, or by the addition of pharmaceutically acceptable acid salts. These salts include salts of mineral acids, for example, hydrochloric acid, sulfuric acid, nitric acid and the like; salts of monobasic carboxylic acids, such as, for example, acetic acid, propionic acid and the like; salts of dibasic carboxylic acids, such as, for example, maleic acid, fumaric acid, tartaric acid, succinic acid and the like; and salts of tribasic carboxylic acids, such as, for example, carboxysuccinic acid, citric acid and the like.

Preferred pharmaceutically acceptable basic addition salts include salts of alkali metals, e.g. sodium or potassium; alkaline earth metals, e.g., calcium or magnesium; or complex salts, e.g., ammonium or substituted ammonium salts such as mon-, di- or trialkylammonium salts or mono, di- or trihydroxyalkylammonium salts.

The cellulosic polymers of the formulations and sustained/prolonged release dosage forms of-the present invention comprise glucose polysaccharide ethers having multiple glucose units and methyl, ethyl, hydroxyethyl, hydroxypropyl or hydroxypropyl methyl substitution. Exemplary cellulosic polymers having methylether substitution are the Methocel series, i.e., Methocel E, Methocel A, Methocel K, Metoloses and the Ethocels, for example, Ethocel P20 and low-substituted hydroxpropyl ether cellulose polymers, LH Series.

The formulations of the invention can be administered orally, for example, with an inert diluent, coated or encapsulated, including finished matrix tablets contained in a capsule. Typically the formulation is manufactured by using conventional blending or granulating, milling, and/or sieving followed by tabletting. Granulation is used to achieve a particle size, improve homogeneity and reduce adherence. Coating may occur after granulation or tabletting.

For the purpose of oral therapeutic administration, the compounds can be incorporated with excipients and also used in the form of troches, capsules, chewing gums, as well as tablets, capsules, and caplets. These preparations should contain at least 0.5% of active compound, but the amount can be varied depending upon the particular form and can conveniently be between 4.0% to about 70% of the dosage form.

Tablets, pills, capsules, troches, and the like can contain the following ingredients: a binder, such as starch, polyvinyl pyrrolidone, gum tragacanth or gelatin; a filler, such as microcrystalline cellulose or lactose; a disintergrating agent, such as crospovidone, sodium starch glycolate, corn starch, and the like; a lubricant, such as magnesium stearate, stearic acid, glyceryl behenate; a glidant, such as colloidal silicon dioxide and talc; a sweetening agent, such as sucrose or saccharin, aspartame, acesulfame-K; or flavoring agent, such as peppermint, methyl salicylate, or orange flavoring. When the dosage unit form is a capsule, it can contain, in addition to material of the above type, a liquid carrier, such as a fatty oil.

Other dosage unit forms can contain other materials that modify the physical form of the dosage, for example, as coatings. Thus, dosage forms, for example tablets and capsules, can be coated with sugar, shellac, sustained and other enteric coating agents. Materials used in preparing these compositions should be pharmaceutically pure and nontoxic in the amounts used.

It is to be understood, however, that for any particular subject, specific dosage regimens should be adjusted to the individual need and the professional judgement of the person administering of the formulations of the invention. It is to be further understood that any particular dosage set forth herein are exemplary only and that they do not, to any extent limit the scope or practice of the invention.

Surfactants, which may optionally be employed with the oral formulations, e.g. tablets of the present invention, comprise polysorbates, such as ethers of polyoxyethylene sorbitan and fatty acids. Exemplary surfactants are polysorbate 80 and polyoxyethylene 20 sorbitan monoleate, polyoxyxethylene alkyl ethers of the Brig- or Volpo series, Cremophor RH, Cremophor E1, polyoxethylene sorbitant fatty acid esters of the Tween- or Crillet series, polyoxyethylene stearates of the Cerosynt- or Myrj series, lecithin, poloxamers, d-2 -tocophenyl polyethylene glycol 1000 succinate (Vitamin E TPGS) and saturated polyglycolized glycerides (Labrosol, Labrafile and Gelucires), polysorbate 80 being preferred.

As indicated above, the formulations or constructs of the present invention may contain other various materials which modify the physical form of the dosage form (the subject construct), for example, as coatings, or tablet(s) contained within a capsule. Thus, particles of the subject controlled release formulation of the present invention may be coated with sugar, shellac, sustained and other enteric coating agents. Materials used in preparing these various compositions should be pharmaceutically pure and non-toxic in the amounts used.

In a variation of the above alternative embodiment, the resultant construct is treated whereby only the top surface area thereof has a shell coating thereon. In this regard, reference is made to U.S. Pat. No. 5,916,584, incorporated hereinto by reference in its entirety, which describes the process for forming such a shell. The resulting formulation is one which provides a delay time prior to release of the water soluble active ingredient or ingredients to the patient being treated.

It is to be understood that the medicament, e.g. water soluble medicament, can be employed in the formulation alone or combined with another medicament. The amount of medicament or medicaments is one which is sufficient to therapeutically treat a disease state in the patient being treated thereby.

The term “amount” as used herein refers to a quantity or a concentration as appropriate to the context. The amount of drug that constitutes a therapeutically effective amount varies according to factors such as the potency of the particular biotherapeutic medicament, the route of administration of the formulation and the mechanical system used to administer the formulation. A therapeutically effective amount of a particular drug or combination of drugs can be selected by those of ordinary skill in the art with due consideration of such factors. Generally, a therapeutically effective amount of a biotherapeutic medicament in tablet form for oral administration will be from about 1.0 mg to about 300 mg of the active ingredient or medicament.

The formulation of the water soluble medicaments, of the present invention is useful for the treatment, e.g. by oral administration, of various diseases and disorders, for example, hydroxyzine hydrochloride as an anxiolytic or antihistamine, metoprolol as an antihypertensive or anti-anginal agent, niacin (nicotinic acid) as a vitamin enzyme cofactor or lipid altering agent, caff-eine as a central nervous system stimulant, theophylline as a bronchodilator, diltiazem as an anti-anginal agent, albuterol as a bronchodilator, metronidazole as an antibacterial, metochlopramide as an anti-emetic, captopril as an antihypertensive, nefazodone as an antidepressant agent, zolpidem as an anxiolytic agent, sertraline as an antidepressant agent, labetalol and atenolol as antihypertensive agents. The drugs are readily available from commercial suppliers.

Typically, the sustained/prolonged release pharmaceutical unit dosage forms are prepared by several processes, including but not limited to direct compression (direct blending of ingredients followed by compression), modified direct compression (partial granulation followed with direct blending and compression), and wet granulation (wet mass and blending of all excipientg followed by compression). All finished dosage forms can be followed with a combination of rapid enteric and/or sustained coating systems.

We claim:

1. A sustained/prolonged release pharmaceutical formulation comprising:
(a) a water soluble medicament and (b) a polymer mixture comprising a first component comprising about 80 weight percent of polyvinyl acetate combined with about 20 weight percent polyvinyl pyrrolidone of the total weight of said first component, combined with a second component comprising a cellulose ether polymer.

2. A pharmaceutical unit dosage form according to claim 1 wherein said first component is present in an amount ranging from about 20 weight percent to about 90 weight percent of the total formulation and said second component ranges from about 2 weight percent to about 60 weight percent of the total weight of the formulation.

3. The formulation according to claim 2 wherein said cellulose ether polymer is selected from the group consisting of methyl-, ethyl-, hydroxyethyl-, hydroxypropyl- or hydroxypropyl methyl—substituted polymers of Methocel A series; hydroxypropyl methyl celluloses of METHOCEL E, F, J, or K series at various viscosity grades; different viscosity grades of hydroxy proplyl celluloses of Klucel, or Methocel series; a low substituted grades of hydroxypropyl celluloses of the LH series, and ethyl celluloses of ETHOCEL P series, or a mixture of any of the foregoing ethers.

4. The formulation according to claim 3 wherein said water soluble medicament is selected from the group consisting of a pharmaceutically acceptable addition salt of hydroxyzine, a pharmaceutically acceptable addition salt of metoprolol, niacin, caffeine, theophylline, a pharmaceutically acceptable acid addition salt of diltiazem, a pharmaceutically acceptable acid addition salt of albuterol, a pharmaceutically acceptable acid addition salt of metformin, a pharmaceutically acceptable acid addition salt of metronidazole, a pharmaceutically acceptable acid addition salt of metolopramide, a pharmaceutically acceptable acid addition salt of ranitidine, a pharmaceutically acceptable acid addition salt of captopril, a pharmaceutically acceptable acid addition salt of nefazodone; a pharmaceutically acceptable acid addition salt of zolpidem; a pharmaceutically acceptable acid addition salt of sertraline; a pharmaceutically acceptable acid addition salt of labetalol; and a pharmaceutically acceptable acid addition salt of atenolol.

5. A pharmaceutical construct comprising:
(a) a water soluble medicament;
(b) a polymer mixture comprising (a′) a first component comprising about 80 weight percent of polyvinyl acetate combined with about 20 weight percent polyvinyl pyrrolidone of the total weight of said first component; combined with (b′) a second component comprising a cellulose ether polymer.

6. The pharmaceutical construct as defined in claim 5, wherein said first component is present in an amount ranging from about 20 weight percent to about 90 weight percent of the total formulation and said second component ranges from about 2 weight percent to about 60 weight percent of the total weight of the formulation

7. The pharmaceutical construct as defined in claim 6 wherein said cellulose ether is selected from the group consisting of methyl cellulose, hydroxy propyl cellulose, hydroxy propyl methyl cellulose METHOCEL A series, METHOCEL E series, METHOCEL F series, METHOCEL K series, Metoloses LH series, ETHOCEL P series, or a mixture of any of the foregoing cellulose ethers.

8. The pharmaceutical construct as defined in claim 7 wherein said water soluble medicament is selected from the group consisting of a pharmaceutically acceptable addition salt of hydroxyzine, a pharmaceutically acceptable addition salt of metoprolol, niacin, caffeine, theophylline, a pharmaceutically acceptable acid addition salt of diltiazem, a pharmaceutically acceptable acid addition salt of albuterol, a pharmaceutically acceptable acid addition salt of metformin, a pharmaceutically acceptable acid addition salt of metronidazole, a pharmaceutically acceptable acid addition salt of metoclopramide, a pharmaceutically acceptable acid addition salt of ranitidine, a pharmaceutically acceptable acid addition salt of captopril, a pharmaceutically acceptable acid addition salt of nefazodone; a pharmaceutically acceptable acid addition salt of zolpidem; a pharmaceutically acceptable acid addition salt of sertraline; a pharmaceutically acceptable acid addition salt of labetalol; and a pharmaceutically acceptable acid addition salt of atenolol.

9. A process for the preparation of a sustained/prolonged release pharmaceutical unit dosage form comprising the steps of:
(a) fluidizing a water soluble medicament combined with a carrier, comprising a polymer mixture comprising a first component, comprising about 80 weight percent of polyvinyl acetate combined with about 20 weight percent of polyvinyl pyrrolidone of the total weight of said first component, combined with a second component comprising a cellulose ether polymer; to form a fluidized mixture;
(b) direct blending the mixture to form a blended mixture;
(c) granulating said blended mixture to form a granulated mixture; and
(d) tabletting said granulated mixture and/or blend to form a tablet.

10. The process according to claim 9 wherein said water soluble medicament is selected from the group consisting of a pharmaceutically acceptable addition salt of hydroxyzine, a pharmaceutically acceptable addition salt of metoprolol, niacin, caffeine, theophylline, a pharmaceutically acceptable acid addition salt of diltiazem, a pharmaceutically acceptable acid addition salt of albuterol, a pharmaceutically acceptable acid addition salt of metformin, a pharmaceutically acceptable acid addition salt of metronidazole, a pharmaceutically acceptable acid addition salt of metoclopramide, a pharmaceutically acceptable acid addition salt of ranitidine, a pharmaceutically acceptable acid addition salt of captopril, a pharmaceutically acceptable acid addition salt of nefazodone; a pharmaceutically acceptable acid addition salt of zolpidem; a pharmaceutically acceptable acid addition salt of sertraline; a pharmaceutically acceptable acid addition salt of labetalol; a pharmaceutically acceptable acid addition salt of atenolol.

11. A modulated release pharmaceutical construct which comprises a matrix of a water soluble medicament associated with a polymer mixture, where said mixture comprises a first component, comprising about 80 weight percent of polyvinyl acetate combined with about 20 weight percent of polyvinyl pyrrolidone of the total weight of said first component, combined with a second component comprising a cellulose ether polymer and a medicament associated with said matrix.

12. A sustained release pharmaceutical composition comprising a construct comprising a water soluble medicament and a polymer mixture, comprising a first component comprising about 80 weight percent of polyvinyl acetate combined with about 20 weight percent of the total weight of said first component of polyvinyl pyrrolidone, combined with a second component comprising a cellulose ether polymer.

13. A process for preparing a sustained/prolonged release pharmaceutical unit dosage form, which comprises:
(a) blending a water soluble medicament with a polymer mixture comprising a first component comprising about 80 weight percent ofpolyvinyl acetate combined with about 20 weight percent of polyvinyl pyrrolidone of the total weight of said first component, combined with a second component comprising a cellulose ether polymer, to form a mixture; and
(b) tabletting said mixture

14. The process as defined in claim 13 wherein the tabletting is conducted under direct compression.

15. The process as defined in claim 13 wherein said polymer and drug are blended by means of wet granulation followed by dry blending.

16. The process as defined in claim 13 wherein all materials are wetted prior to said blending and dried and milled after said blending.
